(19) United States
(12) Patent Application Publication
(10) Pub. No.: US 2019/0249363 A1
(43) Pub. Date: Aug. 15, 2019

(54) METHOD FOR EXTRACTING CELLUSOSE, HEMICELLULOSE AND LIGNIN FROM LIGNOCELLULOSE FROM PLANT BIOMASS

(71) Applicant: Clariant International Ltd., Muttenz (CH)

(72) Inventors: Manuela FRICK, Pfronten (DE); Franz EFFENBERGER, Stuttgart (DE)

(73) Assignee: Clariant International Ltd., Muttenz (CH)

(21) Appl. No.: 16/340,669

(22) PCT Filed: Oct. 6, 2017
(86) PCT No.: PCT/EP2017/075441

§ 371 Date: Apr. 9, 2019

(30) Foreign Application Priority Data
Oct. 11, 2016 (DE) 10 2016 219 719.3

(51) Int. Cl. D21C 1/02; C12P 7/10; D21C 1/06; D21C 9/14; D21C 1/04; D21C 9/153; D21C 9/16

(52) U.S. Cl. CPC D21C 1/02; C12P 7/10; D21C 1/06; D21C 9/144; C12P 2203/00; D21C 9/153; D21C 9/163; D21C 9/166; C12P 2201/00; D21C 1/04

(57) ABSTRACT

A process for obtaining cellulose, hemicellulose and lignin from lignocellulose can be carried out in an environmentally friendly manner when it comprises the following steps:
step a) wherein a lignocellulose from plant biomass is provided,
step b) wherein the lignocellulose is brought into contact with a first mixture M1 containing water and an alkaline component, and a first suspension S1 forms, the first suspension S1 comprises a first solid F1 and a first liquid phase P1, wherein the first solid F1 contains a crude cellulose, and the first liquid phase P1 contains hemicellulose and lignin, and
step c) wherein the crude-cellulose-containing solid F1 is brought into contact with a second mixture M2 containing formic acid and water and optionally acetic acid, and a second suspension S2 forms, the second suspension S2 comprises a second solid F2 and a second liquid phase P2, wherein the second solid F2 contains a pure cellulose, and the second liquid phase P2 contains hemicellulose and lignin.

The growing world population and the higher requirement for foodstuffs necessarily associated therewith, but also the drastically increased energy requirement in the developed countries, are compelling humanity to protect the world's existing resources. It is therefore an aim to obtain from renewable raw materials those products that were hitherto produced by petrochemical processes from crude oil, natural gas or coal. The basis for renewable raw materials is photosynthesis. Under the influence of the sun's energy, carbon dioxide reacts with water to form glucose and oxygen. This is the prerequisite for the production of foodstuffs which contain, for example, starch, proteins and fats, and also for the storage of energy, for example in the form of cellulose in wood.

In recent decades, the cultivation of cereals in order to obtain bioethanol as a replacement fuel for the transport sector has been greatly promoted. In the meantime, however, it has become accepted opinion that, of the renewable raw materials, those which can serve primarily as foodstuffs, such as cereals and plant oil, should not be used in the energy sector as a fuel. In recent years, efforts have therefore been directed at using primarily bio-waste materials, such as straw and organic waste, for the energy sector. The most important waste materials include the so-called lignocelluloses of annual plants, which are obtained in large amounts as straw in the cultivation of cereals. Although the relative proportions in the different straw types, such as wheat, rice and maize, vary slightly, straw generally contains the main constituents cellulose, hemicellulose and lignin, in most cases approximately from 35 to 45% by weight cellulose, from 30 to 35% by weight hemicellulose and from 15 to 20% by weight lignin. In addition, straw contains smaller amounts of lipids and inorganic substances, in particular silicates.

The use of lignocelluloses from straw hitherto focused on converting the cellulose and, where possible, also the hemicellulose into bioethanol or using the cellulose as pulp for paper production.

According to the functions that are to be fulfilled by, for example, the stalks or stems in the cereal in respect of, for example, strength, moisture regulation and plant protection, lignocelluloses in the straw can be understood as being composite materials which are not accessible for alcoholic fermentation to bioethanol or for the production of pulp without special pulping processes.

In order to obtain pulp from straw, comparable pulping processes are used as in the production of pulp from lignocelluloses of perennial plants such as trees, these also include alkaline pulping, which is also referred to as soda pulping or the soda process. In woods, however, only incomplete pulping is achieved by the soda process.

The pulping of straw, which is also referred to as “pretreatment”, is generally carried out under drastic conditions in order to obtain bioethanol. For example, so-called “steam explosion pulping” is carried out under elevated pressure and at 160° C. to 230° C. In alkaline pulping, temperatures of from 85° C. to 180° C. are used. Mineral acids are frequently used as catalyst. Conventional soda pulping takes place at temperatures of from 160° C. to 170° C. for several hours and with alkali concentrations of from 12% by weight to 20% by weight, based on biomaterial used.

In R. Rinaldi (Aufschluss pflanzlicher Biomasse trifft auf Katalyse, Angewandte Chemie, 2014, 126, 8699-8701), an overview of pulping methods is given. Lignocelluloses are regarded as core-shell composites, wherein the cellulose, as the core, is protected by a more reactive shell of lignin and hemicelluloses. The shell of lignin and hemicelluloses is detached from the cellulose matrix by acid- or base-catalyzed solvolysis. Cellulose and hemicelluloses are isolated either as polymers or as degradation products, such as monosaccharides. Lignin remains as a degraded polymer residue in a collapsed matrix.

However, lignin can also be understood as being a filling material which makes a decisive contribution to the strength of the plant only indirectly and is at least in part not chemically bonded to the cellulose.

In N. Sarkar et al. (Bioethanol production from agriculture wastes: an overview, Renewable Energy, 37, 2012, 19-27) there are presented physical, physicochemical, chemical and biological processes for pretreating biomass which precede an enzymatic hydrolysis or fermentation.

WO 2006/111604 describes the alkaline pulping of lignocelluloses, wherein a crude cellulose obtained in the alkaline pulping is ground in the wet state. The separation of hemicelluloses is improved by adding surface-active substances to the alkaline solution. Crude cellulose, which is obtainable after the alkaline pulping, contains considerable amounts of hemicelluloses and lignin as well as relatively large amounts of biomass which has not been pulped (fragments).

In G.-H. Delmas et al. (Functionality of wheat straw lignin extracted in organic acid media, Journal of Applied Polymer Science, 121, 2011, 491-501), lignin and hemicelluloses are extracted from wheat straw with an organic-acid-containing extracting agent. Hemicelluloses are thereby degraded to pentoses.

U.S. Pat. No. 7,402,224 relates to a process for producing pulp, lignins, sugars and acetic acid. Hemicelluloses and lignins are hydrolyzed.

H. Q. Lam et al. (A new procedure for the destructuring of vegetable matter at atmospheric pressure by a catalyst/solvent system of formic acid/acetic acid. Applied to the pulping of triticale straw, Industrial Crops and Products 14, 2001, 139-144) disclose the separation of cellulose, hemicelluloses and lignin from triticale straw at atmospheric pressure in an aqueous medium by the so-called organosolv process).

B. Benjelloun (Tomorrow's biorefineries in Europe, The CIMV Organosolv Process, presentation in Brussels 11-12 Feb. 2014) presents a variant of the organosolv process, which yields lignin, cellulose pulp and C₅ sugars. When the organosolv process is carried out, hemicellulose is degraded to low molecular weight oligomers or monomers. Lignin is no longer in globular form but in linear form (Guo-Hua Delmas, Bouchra Benjelloun-Mlayah, Yves Le Bigot, Michel Delmas, Functionality of wheat straw lignin extracted in organic acid media, Journal of Applied Polymer Science, 121, 2011, 491-501). In addition, the cellulose obtained by the organosolv process contains very large amounts of silicates, which are not acceptable for uses in the food sector.

In K. Salehi et al. (Comparison of MEA/AQ, soda and soda/AQ pulping of wheat and rye straw, Industrial Crops and Products, 52, 2014, 603-610), known processes for pulping lignocelluloses from bio-waste such as straw are summarized. The effectiveness of aqueous monoethanolamine solution as an extracting agent is shown.

WO 2015/075080 describes the isolation of lignin from biomass containing lignocellulose, wherein a mixture of water and at least one organic solvent is used.

W. O. S. Doherty et al. (Value-adding to cellulosic ethanol: Lignin polymers, Industrial Crops and Products, 33, 2011, 259-276) describe a process for extracting lignin from lignocellulose, such as the sulfite process, the kraft process and the soda process, and give an overview of possibilities for using the extracted lignin.

U.S. Pat. No. 6,503,369 relates to a process for producing cellulose and fertilizers, wherein pulp is bleached and process water and chemicals used are thereby recycled.

In J. Beringer (Zellstoff aus Weizenstroh: Gewinnung durch Aufschlussverfahren mit Ameisen-und Essigsäure sowie Untersuchungen zur Zellstoffstruktur und Eignung als Paper-und Chemiezellstoff, dissertation 2004, University of Stuttgart), the suitability of a pulping process based on a mixture of formic acid and acetic acid and of a pulping process based on monoethanolamine for obtaining pulp from wheat straw and other annuals is studied.

The use of annual plants such as straw has been investigated and optimized especially as regards the production of bioethanol and the obtainment of pulp. The two other main components of lignocellulose, hemicellulose and lignin, are generally considered to be less important. Thus, when obtaining bioethanol, an attempt is also made to convert the hemicelluloses into bioethanol by special enzyme systems. The lignin that is obtained is generally used thermally, that is to say burned.

A disadvantage of the known processes is inter alia that the hemicelluloses are to a large extent degraded to sugars, such as pentoses.

Moreover, in the case of acetic acid-formic acid pulping, for example, the lignin is frequently no longer in the original globular form but is likewise degraded to linear fragments. Furthermore, many of the known process variants represent a considerable burden on the environment as a result of their emissions.

It is an object of the present invention to avoid the disadvantages of the known processes and to provide a process which allows cellulose, hemicellulose and lignin to be separated in high yield and high purity starting from lignocellulose.

The object is achieved by a process for obtaining cellulose, hemicellulose and lignin from lignocellulose, which process comprises:
a step a) wherein a lignocellulose from plant biomass is provided,
a step b) wherein the lignocellulose is brought into contact with a first mixture M1 containing water and an alkaline component, in particular sodium hydroxide, and a first suspension S1 forms, the first suspension S1 comprises a first solid F1 and a first liquid phase P1, wherein the first solid F1 contains a crude cellulose, and the first liquid phase P1 contains hemicellulose and lignin, and
a step c) wherein the crude-cellulose-containing solid F1 is brought into contact with a second mixture M2 containing formic acid and water and optionally acetic acid, and a second suspension S2 forms, the second suspension S2 comprises a second solid F2 and a second liquid phase P2, wherein the second solid F2 contains a pure cellulose, and the second liquid phase P2 contains hemicellulose and lignin.

The described process is a mild pulping process which is suitable for decomposing lignocellulose into its main constituents cellulose, hemicellulose and lignin and for isolating those constituents in high purity and with good yields so that they can be used for known and novel applications. The object is achieved substantially by combining step b) with step c), wherein step b) is carried out before step c).

In step b), more than 70% by weight of the lignin contained in the lignocellulose and also a portion of the hemicellulose, in each case in non-degraded form, are dissolved out.

In step c), further lignin and also further hemicellulose is removed from the crude cellulose. In addition, in step c), residues of the plant biomass which have not yet been pulped or have been only insufficiently pulped, which residues are also referred to as fragments and may still be contained in the crude cellulose, are also pulped. The amount of unusable waste, which is also referred to as “reject”, is thereby reduced or such waste is avoided altogether.

The pure cellulose obtained by the process according to the invention can be subjected to a bleaching operation to a reduced extent, in particular with a reduced number of bleaching stages. Furthermore, the crude cellulose is of improved quality in particular as regards a degree of whiteness (89 according to Hunter) and a proportion of α-cellulose (98%).

The plant biomass is preferably from monocotyledons, in particular from grasses such as wheat, barley, rye, oats, triticale, rice, millet, sugar cane, maize, bamboo, Chinese silver grass, etc. The plant biomass used particularly preferably consists of straw, for example wheat straw.

Lignocellulose is a complex composite material of cellulose, hemicellulose, lignin and minerals, such as, for example, silicates.

Cellulose is a linear polymer composed of β-D-glucopyranoses which are linked with one another by 1,4-β-glycosidic linkages, which means that the anhydroglucose units [AGU] are alternately rotated through 180°, so that the repeating unit of cellulose is the disaccharide cellobiose. The degree of polymerization is given as from 1000 to 15000 anhydroglucose units. Cellulose forms highly ordered structures via intra- and inter-molecular hydrogen bridges. The proportion of crystalline regions is from 70% to 80%. The properties of cellulose (tear strength, solubility, swellability, etc.) are determined mainly by the supramolecular structure. The α-cellulose content refers to a high molecular weight fraction of cellulose which is insoluble in 17.5% strength sodium hydroxide solution. The α-cellulose content is usually determined in accordance with IPS testing method TAPPI T 203 cm-99.

Hemicelluloses are polyoses and a group of polysaccharides which differ from cellulose in that a plurality of molecules are not only composed of a single sugar structure (glucose) but consist of different sugars and contain additional functional groups. They can also comprise branched and less long molecule chains and have a lower average degree of polymerization, at from 50 to 250, than cellulose. The sugar components of the polyoses are generally divided into the groups pentoses, hexoses, hexuronic acids and deoxy-hexoses.

Lignin is a highly crosslinked polymer composed of phenylpropane units. The three most important basic components are phenylpropane derivatives. They can be divided into the syringyl type, the guaiacyl type and the p-hydroxyphenyl type.

By means of the first mixture M1, the major amount of lignin is dissolved out of the lignocellulose. Ester bonds of hemicellulose to cellulose, or to lignin, are also cleaved. In addition, a large part of the silicates contained in the lignocellulose is separated from the cellulose in the form of soluble sodium compounds.

The first mixture M1 preferably comprises from 1% by weight to 30% by weight sodium hydroxide and from 70 to 99% by weight water, in particular from 2 to 5% by weight sodium hydroxide and from 95 to 98% by weight water, based on the first mixture M1. A higher content of sodium hydroxide in the first mixture M1 leads to a higher yield of hemicellulose, whereby the yield of crude cellulose falls.

In step b), a first ratio MV1 (dry mass in g/volume in ml) of lignocellulose used to first mixture M1 used is preferably from 1:3 to 1:15, in particular from 1:7 to 1:10.

Preferably, after step b), in a step b1), the first solid F1 is separated from the first liquid phase P1, and a first precipitating agent FM1, in particular ethanol, is added to the first liquid phase P1, whereby at least portions of the hemicellulose are precipitated and a first, hemicellulose-containing precipitate thus forms in a mixture G1. Particularly preferably, the first solid F1 is separated from the first liquid phase P1 by means of filtration.

In step c), hemicellulose and smaller amounts of lignin still present, which are presumably bonded to the cellulose by acetal or ether bonds, are separated from the crude cellulose. Our own investigations have shown that these acetal and/or ether bonds cannot be cleaved using dilute mineral acids since condensation reactions occur. Organic acids, on the other hand, such as acetic acid and formic acid, are suitable for that purpose. Such acids are biodegradable, non-toxic and environmentally acceptable.

By means of the second mixture M2, the crude cellulose is separated into pure cellulose, hemicellulose and lignin, and in particular fragments still present are pulped.

The second mixture M2 can consist (also only) of formic acid and water.

Preferably, the second mixture M2 comprises more than 50% by weight formic acid, in particular when the second mixture M2 consists of formic acid and water. Further preferably, the second mixture M2 comprises not less than 77% by weight formic acid. Preferably, the second mixture M2 comprises not more than 23% by weight water.

In one embodiment, the second mixture M2 has a mass ratio [g/g] of formic acid to acetic acid of from 4:1 to 2:1, more preferably from 3.2:1 to 2.8:1, particularly preferably 3:1.

Preferably, in step c), the second mixture M2 comprises from 0% by weight to 40% by weight acetic acid, from 40% by weight to 100% by weight formic acid and from 0% by weight to 40% by weight water; preferably from 0.1% by weight to 40% by weight acetic acid, from 40% by weight to 80% by weight formic acid and from 10% by weight to 40% by weight water; in particular from 15% by weight to 25% by weight acetic acid, from 55% by weight to 65% by weight formic acid and from 15% by weight to 25% by weight water, based on the second mixture M2, for example 60.4% by weight formic acid, 20.4% by weight acetic acid and 19.2% by weight water, or 51.8% by weight formic acid, 17.5% by weight acetic acid and 30.7% by weight water. A lower water content leads to an improved yield of lignin and an improved bleaching action as regards the cellulose.

Preferably, in step c), the second mixture M2 is in the form of an azeotropic mixture. The use of the second mixture M2 in the form of an azeotropic mixture is advantageous because it can easily be removed from the resulting second liquid phase P2 by distillation and recycled.

Preferably, in step c), a second ratio MV2 (dry mass in g/volume in ml) of crude-cellulose-containing solid F1 used to second mixture M2 used is from 1:5 to 1:30, in particular from 1:15 to 1:25.

Preferably, after step c), in a step c1), the second solid F2 is separated from the second liquid phase P2, and the second liquid phase P2 is subjected to precipitation, wherein a second precipitating agent FM2, in particular water, is optionally added to the second liquid phase P2; whereby at least portions of the lignin are precipitated and a second, lignin-containing precipitate forms in a second mixture G2. Particularly preferably, the second solid F2 is separated from the second liquid phase P2 by means of filtration.

Preferably, after step b1), in a step b2), the first, hemicellulose-containing precipitate is separated from the first mixture G1, the first precipitating agent FM1 is removed at least in part from the first mixture G1, a third precipitating agent FM3, in particular hydrochloric acid, is added to the first mixture G1, which is depleted in respect of the first precipitating agent FM1, and at least a portion of the lignin is precipitated. Particularly preferably, the first, hemicellulose-containing precipitate is separated from the first mixture G1 by means of filtration.

Preferably, after step c1), in a step c2), the second, lignin-containing precipitate is separated from the second mixture G2, and the second mixture G2 is so dried that hemicellulose is present in concentrated form, in particular in the form of a viscous sugar syrup. Particularly preferably, the second, lignin-containing precipitate is separated from the second mixture G2 by means of filtration.

Preferably, step b) and/or step c) are carried out at a pressure of from 0.8 bar to 1.5 bar, in particular from 0.9 bar to 1.2 bar.

Preferably, step b) is carried out at a first temperature T1 and step c) is carried out at a second temperature T2, wherein the first temperature T1 and/or the second temperature T2 are from 20° C. to 150° C., in particular from 70° C. to 125° C.

Preferably, in step a), the lignocellulose is provided from comminuted plant biomass, and in particular more than 90% by weight of the comminuted plant biomass, based on dry comminuted plant biomass, comprises particles having a maximum spatial extent of from 0.5 mm to 20 mm, in particular from 1 mm to 5 mm. Comminution preferably takes place by grinding.

Preferably, prior to step b), the lignocellulose is brought into contact, in particular extracted, with an alcohol, in particular ethanol, and at least a portion of the alcohol is removed again prior to step b). Preferably, the alcohol is removed by distillation. After the alcohol has been removed by distillation, approximately 2% by weight lipids, based on the straw used, are usually obtained. The lignocellulose is preferably not dried before step b) is carried out.

The pure cellulose obtained is preferably subjected to a bleaching process, wherein possible bleaching processes are sodium chlorite bleaching, bleaching with hydrogen peroxide, bleaching with peracetic acid and/or bleaching with ozone. Also preferably, the bleaching process is carried out with a bleaching sequence comprising sodium chlorite bleaching (CT), alkaline extraction (E) and a second sodium chlorite bleaching (CT), in particular in the sequence CT-E-CT.

The invention relates additionally to lignin obtained from a first liquid phase P1 and/or from a second liquid phase P2 by the process according to the invention.

The invention is explained in greater detail by the following FIGURE, the examples and the claims.

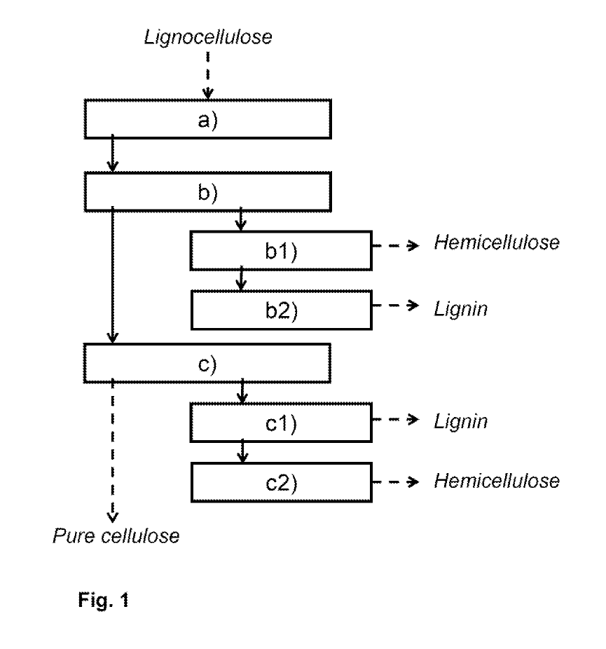

The invention is illustrated with the aid of the FIGURE (FIG. 1), which shows an example of an embodiment of the process according to the invention. Lignocellulose is used and pure cellulose, hemicellulose and lignin are obtained by carrying out steps a), b), b1), b2), c), c1) and c2), wherein pure cellulose is obtained in the form of a solid in step c), hemicellulose is obtained in the form of a solid, or syrup, in steps b1) and c2), and lignin is obtained in the form of a solid in steps b2) and c1).

### EXAMPLE

Step a)

Wheat straw was finely ground to a particle size of about 2 mm. The ground wheat straw, which had a moisture content corresponding to ambient humidity, was subjected to extraction with ethanol. The extracted wheat straw could be processed without being dried. In order to determine exact yields, the straw was dried at 60° C. after the ethanol extraction and stored at room temperature. 20 g of dry wheat straw, comprising 0.6 g of dry extract, were present. The sum of dry wheat straw and dry extract is regarded as the starting amount in respect of the mass used in the mass balance.

After the dried wheat straw had been stored under ambient conditions, the swollen wheat straw had a moisture content of 5% by weight, so that 20.4 g of air-dry wheat straw were present.

Step b)

The air-dry wheat straw from step a) having a dry mass of 19.4 g was heated for one hour in a 3-necked flask, with KPG stirring, with 194 ml of a solution of 3.5% strength (g of NaOH/ml of water) sodium hydroxide solution as a first suspension S1 having a ratio, which is also referred to as the liquor ratio, of 1:10 (dry mass in g/volume in ml). The temperature of the 3-necked flask was adjusted to 120° C. by means of a preheated oil bath. The temperature of the first suspension S1, after a short heating phase, was 98° C.

The first suspension S1 was then filtered in vacuo, and the residue was washed with 100 ml of the 3.5% strength sodium hydroxide solution or with 100 ml of water. The filtrate contained dissolved hemicelluloses and lignin.

Crude cellulose with a dry mass of 10.7 g was obtained, which corresponds to 53.5% by weight of the starting amount of 20 g of straw. The composition is indicated in Table 1.

Step b1)

230 ml of ethanol were added to 230 ml of the filtrate from the filtration in step b), which had a pH of 13.2, in order to precipitate hemicelluloses. The mixture was stirred for 15 minutes and then filtered, the resulting filtrate was a first mixture, which contained lignin. A first precipitate, which contained hemicelluloses, remained as residue. The first precipitate was washed with 50 ml of ethanol and dried in vacuo. The hemicellulose was precipitated in part as the alkali salt, because it is easier to handle in the form of a salt. The yield of alkali salt was 4.1 g, which corresponds to 20.5% by weight relative to the straw used. The composition is indicated in Table 2. A sugar analysis gave a hemicellulose content of 12.5% by weight, based on the straw used.

Step b2)

Ethanol was removed from the first mixture from step b1), which contained lignin, at 40° C. by means of a rotary evaporator, and lignin was precipitated at a pH of 2 by addition of 10% by weight hydrochloric acid, and then filtering was carried out.

The residue, which contained lignin, was washed twice with 100 ml of water having a pH of 2 and dried in vacuo. Lignin with a dry mass of 2.3 g was obtained, which corresponds to 11.5% by weight of the starting amount of 20 g of straw. The composition is indicated in Table 2.

Step c)

To the pressed residue from step b), which consisted of 10.7 g of crude cellulose and water, there were added in a 3-necked flask acetic acid, formic acid and water in such an amount that a second suspension S2 having a ratio of 1:19 (dry mass in g/volume in ml) was obtained and the liquid phase contained 20.4% by weight acetic acid, 60.4% by weight formic acid and 19.2% by weight water and was in the form of an azeotropic mixture having a boiling point of 106° C. The resulting second suspension S2 was heated for one hour, with KPG stirring, and adjusted to a temperature of 120° C. with the aid of a preheated oil bath. The temperature in the second suspension S2 was 106° C.

The second suspension S2 was then filtered in vacuo. The second residue, which contained pure cellulose, was washed again, this time with 400 ml of water. Pure cellulose with a dry mass of 8.8 g was obtained, which corresponds to 44.0% by weight of the starting amount of 20 g of straw. The composition is indicated in Table 1.

Step c1)

For precipitation of lignin, acetic acid, formic acid and water were removed as far as possible from the filtrate of the filtration in step c) at 40° C. by means of a rotary evaporator, whereby a dark syrup remained, which was taken up in 100 ml of water. A pH of 2.2 thereby resulted. The mixture was adjusted to a temperature of 120° C. for one hour by means of a preheated oil bath. Filtration was then carried out. The resulting filtrate formed a second mixture G2, which contained hemicellulose in water.

The residue, which contained lignin, was washed with 50 ml of water and dried in vacuo. Lignin with a dry mass of 0.6 g was obtained, which corresponds to 3.0% by weight of the starting amount of 20 g of straw. The composition is indicated in Table 2.

Step c2)

Water was removed from the second mixture G2 from step c1), which contains hemicellulose, at 40° C. by means of rotary evaporator. The resulting syrup was dried in vacuo. 1.2 g of hemicellulose were obtained in the form of syrup, which corresponds to 6% by weight of the starting amount of 20 g of straw.

Bleaching Process

The pure cellulose from step c) was bleached by a standard bleaching process with three-stage bleaching, with the bleaching sequence CT-E-CT, that is to say with sodium chlorite bleaching (CT), alkaline extraction (E) and a second sodium chlorite bleaching (CT), as is described in greater detail in J. Beringer (Zellstoff aus Weizenstroh: Gewinnung durch Aufschlussverfahren mit Ameisen-und Essigsäure sowie Untersuchungen zur Zellstoffstruktur und Eignung als Papier-und Chemiezellstoff, dissertation 2004, University of Stuttgart). Bleached pure cellulose with a dry mass of 8.4 was obtained, which corresponds to 42.0% by weight of the starting amount of 20 g of straw. The composition is indicated in Table 1.

In total, approximately 75% by weight, based on the straw used, of cellulose, hemicellulose and lignin, in each case in isolated form, were obtained. Taking into account 3% by weight natural wheat straw extract from the extraction with ethanol and approximately 6% by weight mineral substances, especially silicates, this corresponds to a product yield of approximately 84% by weight, based on the straw used.

TABLE 1
Composition of the different celluloses obtained

| | | | | | Bleached pure | |
|---|---|---|---|---|---|---|
| | Crude cellulose | | Pure cellulose | | cellulose | |
| | Analyses based in % by weight on | | | | | |
| | Cellulose | | Cellulose | | Cellulose | |
| | fraction | Straw | fraction | Straw | fraction | Straw |
| Cellulose | 63.1 | 33.8 | 76.2 | 33.4 | 81.0 | 34.0 |
| Hemicellulose | 18.6 | 9.9 | 11.5 | 5.0 | 13.1 | 5.5 |
| Lignin | 5.6 | 3.0 | 2.6 | 1.1 | 0.2 | 0.1 |
| Ash | 2.0 | 1.1 | 0.3 | 0.1 | 0.1 | 0.1 |
| Mass balance | 89.3 | 47.8 | 90.5 | 39.6 | 94.4 | 39.7 |
| Correlation | 99.9 | 53.5 | 100.6 | 44.0 | 99.9 | 42.0 |
| with amounts | | | | | | |
| isolated | | | | | | |

TABLE 2
Composition of lignin and hemicellulose obtained

| | Hemicellulose | | Lignin | | Lignin | |
|---|---|---|---|---|---|---|
| | from step b1) | | from step b2) | | from step c1) | |
| | Analysis based in % by weight on | | | | | |
| | Hemi- | | | | | |
| | cellulose | | Lignin | | Lignin | |
| | fraction | Straw | fraction | Straw | fraction | Straw |
| Cellulose | — | — | not determined | | not determined | |
| Hemicellulose | 61.5 | 12.5 | | | | |
| Lignin | 3.3 | 0.7 | 91.3 | 11.5 | 90.0 | 2.7 |
| Ash | 34.7* | 7.1* | 4.0** | 0.5** | 1.7 | 0.1 |
| Mass balance | 99.5 | 20.3 | 95.6 | 10.9 | 93.3 | 2.8 |
| Correlation | 100.5 | 20.5 | 100.9 | 11.5 | 100.0 | 3.0 |
| with amounts | | | | | | |
| isolated | | | | | | |

*Alkali salts
**Amount varies (0.4-6%), always over 80% silicates

The bleached pure cellulose produced had the characteristics shown in Table 3.

TABLE 3
Characterization of the bleached pure cellulose

| Criterion | Result |
|---|---|
| α-Cellulose content | 98% |
| Degree of whiteness according to Hunter | 89 |
| Degree of polymerization DP_{EWNN} | 1670 |

Within the EU, a content of α-cellulose of at least 92% and an ash content of not more than 0.3% is currently required for use in the food sector, which is achieved by the cellulose produced by the process according to the invention.

1. A process for obtaining cellulose, hemicellulose and lignin from lignocellulose, wherein the process comprises:
a step a) wherein a lignocellulose from plant biomass is provided,
a step b) wherein the lignocellulose is brought into contact with a first mixture M1 containing water and an alkaline component, to form a first suspension S1, the first suspension S1 comprises a first solid F1 and a first liquid phase P1, wherein the first solid F1 contains a crude cellulose, and the first liquid phase P1 contains hemicellulose and lignin, and
a step c) wherein the crude-cellulose-containing solid F1 is brought into contact with a second mixture M2 containing formic acid and water and optionally acetic acid, to form a second suspension S2, the second suspension S2 comprises a second solid F2 and a second liquid phase P2, wherein the second solid F2 contains a pure cellulose, and the second liquid phase P2 contains hemicellulose and lignin.

2. The process as claimed in claim 1, wherein step b) and/or step c) are carried out at a pressure of from 0.8 bar to 1.5 bar.

3. The process as claimed in claim 1, wherein the first mixture M1 comprises from 1% by weight to 30% by weight sodium hydroxide and from 70% by weight to 99% by weight water, based on the first mixture M1.

4. The process as claimed in claim 1, wherein in step b) a first ratio MV1 (dry mass in g/volume in ml) of lignocellulose used to first mixture M1 used is from 1:3 to 1:15.

5. The process as claimed in claim 1, wherein after step b), in a step b1), the first solid F1 is separated from the first liquid phase P1, a first precipitating agent FM1, is added to the first liquid phase P1, whereby at least portions of the hemicellulose are precipitated, and a first, hemicellulose-containing precipitate is thus formed in a mixture C1.

6. The process as claimed in claim 1, wherein in step c) the second mixture M2 comprises from 0% by weight to 40% by weight acetic acid, from 40% by weight to 100% by weight formic acid, and from 0% by weight to 40% by weight water, based on the second mixture M2.

7. The process as claimed in claim 1, wherein in step c) the second mixture M2 is in the form of an azeotropic mixture.

8. The process as claimed in claim 1, wherein in step c) a second ratio MV2 (dry mass in g/volume in ml) of crude-cellulose-containing solid F1 used to second mixture M2 used is from 1:5 to 1:30.

9. The process as claimed in claim 1, wherein after step c), in a step c1), the second solid F2 is separated from the second liquid phase P2, the second liquid phase P2 is subjected to precipitation, wherein a second precipitating agent FM2, is optionally added to the second liquid phase P2; whereby at least portions of the lignin are precipitated and a second, lignin-containing precipitate is formed in a second mixture G2.

10. The process as claimed in claim 5, wherein after step b1), in a step b2), the first, hemicellulose-containing precipitate is separated from the first mixture G1, the first precipitating agent FM1 is removed at least in part from the first mixture G1, a third precipitating agent FM3, is added to the first mixture G1, which is depleted in respect of the first precipitating agent FM1, and at least a portion of the lignin is precipitated.

11. The process as claimed in claim 9, wherein after step c1), in a step c2), the second, lignin-containing precipitate is separated from the second mixture G2, and the second mixture G2 is dried that hemicellulose in concentrated form is present.

12. The process as claimed in claim 1, wherein step b) is carried out at a first temperature T1 and step c) is carried out at a second temperature T2, wherein the first temperature T1 and/or the second temperature T2 is from 20° C. to 150° C.

13. The process as claimed in claim 1, wherein in step a) the lignocellulose is provided from a comminuted plant biomass of particles having a maximum spatial extent of from 0.5 mm to 20 mm.

14. The process as claimed in claim 1, wherein the lignocellulose is brought into contact prior to step b) with an alcohol, and at least a portion of the alcohol is removed again prior to step b).

15. The process as claimed in claim 1, wherein the pure cellulose obtained is subjected to a bleaching process, wherein the bleaching process comprises in particular sodium chlorite bleaching, bleaching with hydrogen peroxide, bleaching with peracetic acid and/or bleaching with ozone.

16. A lignin from a first liquid phase P1 and/or from a second liquid phase P2 prepared according to the process of claim 1.
